(19) United States
(12) Patent Application Publication
(10) Pub. No.: US 2023/0000936 A1
(43) Pub. Date: Jan. 5, 2023

(54) ALLEVIATION OF ITCHING SENSATIONS USING CANNABINOID COMPOUNDS

(71) Applicant: Demetrix, Inc., Berkeley, CA (US)

(72) Inventors: Cynthia W. Bryant, Raleigh, NC (US); Alison Watta, Durham, NC (US)

(21) Appl. No.: 17/855,318

(22) Filed: Jun. 30, 2022

Related U.S. Application Data
(60) Provisional application No. 63/217,266, filed on Jun. 30, 2021.

(51) Int. Cl. A61K 36/185; A61P 17/00; A61K 9/00; A61Q 19/10

(52) U.S. Cl. CPC A61K 36/185; A61P 17/00; A61K 9/0095; A61Q 19/10; A23V 2002/00; A61K 2800/75

(57) ABSTRACT

Methods of treating itching with cannabinoid compounds are described herein. The cannabinoid compounds can include one or more of cannabidol (“CBD”), cannabichromene (“CBC”), cannabigerol (“CBG”), cannabinol (“CBN”), cannabicyclol (“CBL”), cannabidivarin (“CBDV”), cannabigerivarin (“CBGV”), and cannabicyclolic acid (“CBLA”) and variants thereof. The cannabinoid compounds can be antagonists to the receptors CHRM2, EDG3, and MRGPRX2. Compositions and articles including the cannabinoid compounds are further disclosed.

### CROSS-REFERENCE TO RELATED APPLICATIONS

The present application claims the priority benefit of U.S. Provisional Patent App. Ser. No. 63/217,266, entitled “ALLEVIATION OF ITCHING SENSATIONS USING CANNABINOID COMPOUNDS,” filed Jun. 30, 2021, which is hereby incorporated herein by reference in its entirety.

### TECHNICAL FIELD

The present disclosure generally relates to the use of cannabinoid compounds and related products to alleviate the sensation of itching. The cannabinoid compounds can act as antagonists to one or more human receptors that are associated with the sensation of itching.

### BACKGROUND

Itching, sometimes called pruritus, is an uncomfortable sensation characterized by the desire and/or reflex to scratch. Although transmitted by the same neurological signaling pathways as pain, itching is a distinct sensation, and has differing characteristics, than pain. There are several types of itches. For example, itching can be localized or generalized, and can occur as an acute or chronic condition. Chronic itching is defined as itching lasting more than 6 week. Itching can be caused by skin contact with pruritic agents or allergens or by skin conditions including atopic dermatitis or xerosis as well as disorders such as liver and kidney disease. More specifically, pruritoceptive itches originate in the skin due to inflammation, dryness, or other skin damage and is transmitted by unmyelinated C nerve fibers; neuropathic itches are caused by lesions in the afferent pathway; neurogenic itches occurs centrally with an associated neural pathology; and psychogenic itches are caused by mental disorders such as parasitophobia. Neurologically, itching is understood to be mediated by various G protein-coupled receptors (“GPCRs”).

### DETAILED DESCRIPTION

Itching is a disruptive and annoying sensation. Methods and treatments to alleviate the sensation of itching would be highly desirable to improve comfort, concentration, restfulness, and general well-being. Compositions, articles, products, methods and treatments to alleviate, or ameliorate, the sensation of itching using cannabinoid compounds are described herein. In certain embodiments, the cannabinoid compounds described herein can alleviate the sensation of itching by acting as antagonists to one or more of the following GPCR receptors: cholinergic muscarinic receptor 2 (“CHRM2”), sphingosine 1-phosphate receptor 3 (“EDG3”), and MAS-Related GPR Family Member X2 (“MRGPRX2”).

As can be appreciated, activation of the GPCR receptors CHRM2, EDG3, and MRGPRX2 have been identified as being associated with the signaling of the sensation of itching. For example, activation of CHRM2 and MRGPRX2 has been shown to induce itch-associated responses (e.g., through inducement of cholinergic pruritus and the release of endogenous inducers of itching through non-IgE-mediated mast cell degranulation, respectively). Conversely, deactivation and removal of EDG3 receptors has been associated with a reduction in psoriatic itching in mice. Clinical studies are evaluating regulation of these receptors to reduce or treat itching in humans.

The cannabinoid compounds described herein can alleviate, or ameliorate, itching sensations by acting as antagonists to one or more of CHRM2, EDG3, and MRGPRX2. By preventing activation of these specific GPCR receptors, the intensity, duration, type, and/or inducement of associated with the sensation of itching can be alleviated.

Cannabinoid compounds discovered to have an antagonist effect on the identified GPCR receivers include cannabidol (“CBD”), cannabichromene (“CBC”), cannabigerol (“CBG”), cannabinol (“CBN”), cannabicyclol (“CBL”), cannabidivarin (“CBDV”), cannabigerivarin (“CBGV”), and cannabicyclolic acid (“CBLA”) as well as both certain variants thereof such as (+)-cannabidiol (“(+)-CBD”) and certain cannabinoids having alkyl side chains of varying lengths including cannabigerol butyl (“CBG-C4”), cannabigerol hexyl (“CBG-C6”), cannabigerol heptyl (“CBG-C7”), cannabigerol nonyl (“CBG-C9”), cannabidol hexyl (“CBD-C6”), cannabidol heptyl (“CBD-C7”), cannabidol nonyl (“CBD-C9”), and cannabigerolic acid nonyl (“CBGA-C9”).

However, it has also been found that certain cannabinoid compounds do not have an antagonist effect on CHRM2, EDG3, and MRGPRX2 and are not expected to alleviate the sensation of itching since they do not regulate activation of the GPCR receptors. Examples of such cannabinoid compounds which do not exhibit an antagonistic effect on CHRM2, EDG3, and MRGPRX2 include cannabidivarinic acid (“CBDVA”), cannabidiolic acid (“CBDA”), tetrahydrocannabivarin (“THCV”), cannabigerolic acid (“CBGA”), cannabidiorcol (“CBD-C1”), cannabidol-C2 (“CBD-C2”), cannabigerolic acid butyl (“CBGA-C4”), cannabigerovarinic acid (“CBGVA”), cannabigerolic acid hexyl (“CBGA-C6”), cannabigerolic acid heptyl (“CBGA-C7”), and cannabichromenic acid (“CBCA”).

Prior to the present discovery, it was not appreciated which specific cannabinoid compounds could actively influence itching. Prior attempts at addressing concerns associated with itching using traditional cannabis products did not focus the treatment of itching with specific cannabinoid compounds and did not have or rely on the receptor specificity as described in the present disclosure. By selecting only cannabinoid compounds that act upon the GPCR receptors that induce the sensation of itching, the methods, compositions and devices described herein can provide improved alleviation of itching with targeted treatments.

It has additionally been discovered that the alkyl side chain length of cannabinoid compounds can influence the effect the cannabinoid compounds have on the GPCR receptors. For example, it has been discovered that increasing the length of the alkyl side chain, increases the antagonism of CBG to each of CHRM2, EDG3, and MRGPRX2. A similar effect has also been observed for CBGA which in its common form (having a pentyl alkane chain in the 6-position) does not exhibit antagonism to CHRM2, EDG3, and MRGPRX2 while longer alkyl side chains begin exhibiting antagonism with a nonyl alkane chain inhibiting expression of both EDG3 and MRGPRX2. Although trends are observed for the chain length of the cannabinoids, chain length can also be unpredictable as CBD was observed to generally increase in inhibition against CHRM2, EDG3, and MRGPRX2 with increasing chain length, but had an outlier where CBD-C9 did not act as an antagonist to EDG3 while still remaining as an antagonist to CHRM2 and MRGPRX2. CBD-C1 and CBD-C2 having a shorter alkyl side chains, did not exhibit antagonistic effects to any of the receptors.

The present discovery was facilitated by the Applicant's unique methods of producing hereto rare cannabinoid compounds in appreciable quantities including through chemical synthesis reactions and growth in yeast cultures. Prior to the Applicant's research, the lack of viable production of individual cannabinoid compounds obviated the ability to treat itching using only specific cannabinoid compounds. Additional details about the production of producing rare cannabinoid compounds are described in PCT Patent Application Nos. WO 2020/069142 A1, WO 2020/069214 A2, WO 2021/05597 A1; and WO 2020/236789 A1, each of which is incorporated herein by reference.

Prior to the Applicant's process of isolating specific and unique cannabinoid compounds from non-horticultural sources, cannabinoid compounds were extracted and isolated only from naturally grown marijuana plants which drastically limited the volume of the rarer cannabinoid compounds available for research or use. Additionally, cannabinoid compounds with varying chain lengths were entirely unavailable for use prior to the Applicant's formation of such cannabinoids. Thus, these non-horticulturally-derived cannabinoid compounds offer benefits in regard to the alleviation of itching not previously contemplated. As used herein, non-horticulturally derived cannabinoid compounds refers to cannabinoid compounds not grown in plants (e.g., not through horticulture or agriculture).

Additionally, isolated cannabinoid compounds extracted from marijuana plants can also suffer from purity issues as certain unavoidable containments (such as other natural marijuana plant compounds, irremovable amounts of other cannabinoid compounds, etc.) can remain present in isolated cannabinoid compounds extracted from marijuana plants. Such unavoidable containments can impact the quality of the data or even alter the apparent functioning of the cannabinoid compounds. Compositions and methods of alleviating itching that use horticulturally derived cannabinoid compounds may not exhibit the same effects as compositions and methods using purer cannabinoid compounds such as the cannabinoid compounds contemplated herein. As can be appreciated however, horticulturally derived cannabinoid compounds can be used in certain embodiments of the disclosure if the horticulturally extracted cannabinoid compounds are sufficiently pure and/or if any containments are sufficiently well understood.

By acting as antagonists to CHRM2, EDG3, and MRGPRX2, the cannabinoid compounds described herein can alleviate the sensation of itching by mediation of the signaling pathways underlying itching. For example, pruritoceptive itching, neuropathic itching, and neurogenic itching can each be treated by use of the cannabinoid compounds described herein through various embodiments. As can be appreciated, by acting on the receptors, the cannabinoid compounds described herein enjoy wide applicability for the treatment of causes of itching, including chronic itching which has hereto been extremely difficult to treat up until now.

Generally, alleviation of the sensation of itching can be accomplished by treatment with a therapeutically effective amount of one or more of CBD, CBC, CBG, CBN, CBL, CBDV, CBGV, CBLA, (+)-CBD, CBG-C4, CBG-C6, CBG-C7, CBG-C9, CBD-C6, CBD-C7, CBD-C9, and CBGA-C9. In certain embodiments, each of CBD, CBC, CBG, CBN, CBL, CBDV, CBGV, CBLA, (+)-CBD, CBG-C4, CBG-C6, CBG-C7, CBG-C9, CBD-C6, CBD-C7, CBD-C9, and CBGA-C9 can be included in a composition or article to relieve the suffering of itching while in other embodiments, only a subset of CBD, CBC, CBG, CBN, CBL, CBDV, CBGV, CBLA, (+)-CBD, CBG-C4, CBG-C6, CBG-C7, CBG-C9, CBD-C6, CBD-C7, CBD-C9, and CBGA-C9 can be included in such compositions and articles.

For example, in certain embodiments, only one or more of the cannabinoids can be included such as CBC. As can be appreciated however, any combination of CBD, CBC, CBG, CBN, CBL, CBDV, CBGV, CBLA, (+)-CBD, CBG-C4, CBG-C6, CBG-C7, CBG-C9, CBD-C6, CBD-C7, CBD-C9, and CBGA-C9 can be effective including the use of just a single cannabinoid compound selected from the foregoing cannabinoid compounds.

A therapeutic amount of the one or more cannabinoid compounds can vary depending on factors such as the desired effect of treatment, the type of itch being treated (e.g., chronic or a localized itch), the duration of treatment, or the method of delivering the cannabinoid compounds to the subject. For example, to alleviate the sensation of itching of a mosquito bite or other localized skin aliment may require a different amount, or dosage, of the cannabinoid compounds than the amount required to treat chronic itching or widespread itching.

In certain embodiments, a therapeutic amount for internal use (e.g., an oral or injectable composition) can be about 100 mg of the cannabinoid compounds or less; in certain embodiments, about 75 mg of the cannabinoid compounds or less; in certain embodiments, about 50 mg of the cannabinoid compounds or less; in certain embodiments, about 20 mg of the cannabinoid compounds or less; in certain embodiments, about 10 mg of the cannabinoid compounds or less; in certain embodiments, about 5 mg of the cannabinoid compounds or less; in certain embodiments, about 1 mg of the cannabinoid compounds or less; in certain embodiments, about 500 μg of the cannabinoid compounds or less; in certain embodiments, about 100 μg of the cannabinoid compounds or less; and in certain embodiments, about 500 pg of the cannabinoid compounds or less.

For exterior use (e.g., a lotion, cream, bath salt, skin patch, etc.), a therapeutic amount can be relatively higher. For example, in certain embodiments, lotions and creams can include about 2.5 g of the cannabinoid compounds or less; in certain embodiments, about 1.5 g of the cannabinoid compounds or less; in certain embodiments, about 1 g of the cannabinoid compounds or less; in certain embodiments, about 500 mg of the cannabinoid compounds or less; in certain embodiments, about 250 mg of the cannabinoid compounds or less; in certain embodiments, about 100 mg of the cannabinoid compounds or less; in certain embodiments, about 50 mg of the cannabinoid compounds or less; and in certain embodiments, about 10 mg of the cannabinoid compounds or less. Bath salts, being even more diluted, can include, in certain embodiments, about 100 g of the cannabinoid compounds or less; in certain embodiments, about 75 g of the cannabinoid compounds or less; in certain embodiments, about 50 g of the cannabinoid compounds or less; in certain embodiments, about 25 g of the cannabinoid compounds or less; in certain embodiments, about 15 g of the cannabinoid compounds or less; in certain embodiments, about 10 g of the cannabinoid compounds or less; in certain embodiments, about 5 g of the cannabinoid compounds or less; and in certain embodiments, about 1 g of the cannabinoid compounds or less.

As can be appreciated, the relative concentration of the cannabinoid compounds can vary in different compositions and products. For example, a beverage containing the cannabinoid compounds can have a smaller concentration of the cannabinoid compounds than a pill or capsule. In certain embodiments however, the total amount of the cannabinoid compounds can be the same between such two compositions and articles. In other embodiments, both the concentration and amount of cannabinoid compounds can vary between different compositions and articles.

In certain embodiments, the relative amounts of each CBD, CBC, CBG, CBN, CBL, CBDV, CBGV, CBLA, (+)-CBD, CBG-C4, CBG-C6, CBG-C7, CBG-C9, CBD-C6, CBD-C7, CBD-C9, and CBGA-C9 can vary in the compositions and articles described herein. For example, each individual cannabinoid compound (CBD, CBC, CBG, CBN, CBL, CBDV, CBGV, CBLA, (+)-CBD, CBG-C4, CBG-C6, CBG-C7, CBG-C9, CBD-C6, CBD-C7, CBD-C9, and CBGA-C9) can vary from each other cannabinoid compound by about 1,000:1 to about 1:1,000. As can be appreciated, the amount and ratios of each of the cannabinoid compounds can be selected based on factors such as the method of delivery, the type of itch being treated, and individual factors such as the body weight of person consuming the cannabinoid compounds.

In certain embodiments, the compositions, articles, and methods described herein can be substantially or entirely free of cannabinoid compounds other than of CBD, CBC, CBG, CBN, CBL, CBDV, CBGV, CBLA, (+)-CBD, CBG-C4, CBG-C6, CBG-C7, CBG-C9, CBD-C6, CBD-C7, CBD-C9, and CBGA-C9. For example, the compositions, articles, and methods can be substantially or entirely free of CBDVA, CBDA, THCV, CBGA, CBD-C1, CBD-C2, CBGA-C4, CBGVA, CBCA, and tetrahydrocannabinol (“THC”). As used herein, substantially free can mean less than about 5%, less than about 4%, less than about 3%, less than about 2%, less than about 1%, less than about 0.5%, less than about 0.1%, or less than about 0.01%. In certain embodiments, the cannabinoid compounds can be produced using non-horticulturally-derived methods such as through chemical synthesis (e.g., organic synthesis reactions) or through modification of yeast and/or bacterial cells to produce the cannabinoid compounds in high purity. However, in certain embodiments, cannabinoid compounds can also be a natural product, e.g., an extract of a cannabis plant if sufficiently pure. In certain embodiments, substantially pure means that the isolated cannabinoid compound, when added, includes about 3% or less of contaminants, about 2% or less of contaminants, about 1% or less of contaminants, about 0.5% or less of contaminants, about 0.1% or less of contaminants, or about 0.01% or less of contaminants.

In certain embodiments, the compositions, articles, and methods described herein can be utilized on a predetermined schedule (e.g., nightly, twice daily, etc.) or can be utilized on an as-needed basis. In certain embodiments, the predetermined schedule can be based on the half-life of the cannabinoid compounds as well as the release dynamics of the cannabinoid compounds. As can be appreciated, it can be useful in certain embodiments, to release the cannabinoid compounds described herein using a delayed release mechanism, such as a delayed release pill, to regulate the bioavailable amounts of the cannabinoid compounds.

In certain embodiments, the cannabinoid compounds described herein can partially or fully alleviate the sensation of itching by inclusion in a composition or article. The composition or article can be consumed by, or be applied to, a person to alleviate the itching. Generally, the exact nature of the composition or article can vary widely.

For example, to treat itches identified and limited to specific locations, it can be useful in certain embodiments to topically apply the cannabinoid compounds or related composition containing such cannabinoid compounds as described herein. As can be appreciated, if such itch sensations are pruritoceptive itches, it can be useful, in certain embodiments, to provide the cannabinoid compounds in the form of a lotion, gel, liquid, or other personal care composition which can treat both the itching as well as the underlying such as through inclusion of an emollient or moisturizer. In certain embodiments, a topical anesthetic can also be included to treat any accompanying pain or dryness.

In other embodiments, the cannabinoid compounds described herein can be applied topically by immersion in a composition containing the cannabinoid compounds. For example, the water in a bathtub containing the dissolved cannabinoid compounds described herein can therapeutically treat itching. In certain embodiments, the water can be treated by dissolving a solid or liquid product, such as a bath salt or bath soap, containing the cannabinoid compounds. In certain embodiments, such products can additionally contain adjunct ingredients such as clays, oatmeal, baking soda, or other known itching remedies.

Additional examples of personal care compositions include emulsions, suspensions, pastes, ointments, creams, sprays, powders, films, and patches. In certain embodiments, the cannabinoid compounds can be applied to a person through a patch applied to the skin containing the cannabinoid compounds dissolved in a suitable solvent such as an alcohol. The patch can be applied either directly to the location identified by the subject where the sensation of itching is occurring or generally applied to provide general itch alleviation.

In certain embodiments, the cannabinoid compounds can be included in pills or capsules that can be taken quickly and efficiently on a regular or as needed basis (daily, with meals, etc.). Use of an oral pill or capsule can be useful in the treatment of chronic itching. As can be appreciated, pills and capsules can contain a number on inactive ingredients as known in the art such as dicalcium phosphate dehydrate, microcrystalline cellulose, stearic acid, silicon dioxide, croscarmellose sodium, magnesium stearate, and pharmaceutical glaze. Other known pills and capsules are also contemplated herein. As an additional example, a compressed chewable tablet can include a water-disintegrable, compressible carbohydrate (such as mannitol, sorbitol, maltitol, dextrose, sucrose, xylitol, lactose and mixtures thereof), a binder (such as cellulose, cellulosic derivatives, polyvinyl pyrrolidone, starch, modified starch and mixtures thereof), the cannabinoid compounds and, optionally, a lubricant (such as magnesium stearate, stearic acid, talc, and waxes), sweetening, coloring and flavoring agents, a surfactant, a preservative, and other ingredients. All of the ingredients, including the one or more cannabinoid compounds, are dry blended and compressed into a tablet.

In certain embodiments, the cannabinoid compounds can alternatively be administered to individuals via food products and other comestibles. By way of illustration and not as a limitation, the selected cannabinoid compounds can be incorporated into a beverage, a “smoothie” (fruit, vegetable, nut oil, or yogurt based), a frozen desert (e.g., ice cream or sorbet), a food bar, a nutrition bar, a dressing, a snack, into a flour- or flour-alternative-based product, a rice-based product, pastes, gels, powders, gums, etc. Incorporation into food products can facilitate consumption of the cannabinoid compounds and increase palatability.

As can be appreciated, the exact nature of the food article can influence the bioavailability of the cannabinoid compounds. For example, a cannabinoid included in a large food article may take more time to become bioavailable than the same amount of cannabinoid compounds in a single pill or capsule. Generally, the remainder of the composition or article can constitute any suitable non-bioactive component such as filler, food, or water.

In certain embodiments, the compositions or articles including the cannabinoid compounds described herein can include indicia and/or packaging to convey to end users the amount of the cannabinoid compounds contained therein. For example, a small nutrient bar may be individually labeled and packaged to express to the end user that only a single bar should be consumed.

As will be appreciated, a wide variety of different compositions and articles can be prepared which include the one or more cannabinoid compounds of the present disclosure including compositions and articles not listed here. All such compositions and articles are contemplated herein as they are within the ordinary skill of artisans based on the guidance provided in the present disclosure.

Generally, all of the compositions and articles described herein can be manufactured and produced as known in the art. For example, in certain embodiments, the cannabinoid compounds can be dissolved in a suitable solvent such as an alcohol or oil and then added to the composition or article.

### EXAMPLES

To evaluate the role specific cannabinoid compounds play in the neurological signaling pathway for itching, a GPCR reactivity assay was performed to determine the reactivity of 19 cannabinoid compounds to each of CHRM2, EDG3, and MRGPRX2. The evaluated cannabinoid compounds were: CBN, THCV, CBDVA, CBG, CBL, CBC, CBDV, CBDA, CBD, CBGA, (+)-CBD, CBG-C4, CBD-C1, CBD-C2, CBGV, CBGA-C4, CBGVA, CBCA, and CBLA. Acetylcholine was used as the control for CHRM2, sphingosine-1-phosphate was used as the control for EDG3; and cortistatin 14 was used as the control for MRGPRX2.

To perform the GPCR reactivity assay, a commercial GPCR assay, PathHunter® (3-Arrestin from Eurofins DiscoverX Products (Fremont, Calif.), was used. In the PathHunter® (3-Arrestin GPCR assay, an inactive peptide fragment is fused to the targeted GPCR receptor and a complementary peptide fragment is fused to β-arrestin. At activation of the GPCR receptor and recruitment of β-arrestin, complementation of the peptide fragments occurs and restores β-galactosidase activity. The amount of β-galactosidase activity is then measurable using chemiluminescent reagents.

A total of six assays were run. Specifically, agonist and antagonist assays were run for each of CHRM2, EDG3, and MRGPRX2. In each of the three agonist assays, PathHunter® cell lines were removed from a freezer stock and seeded at a volume of 20 μL into white walled, 384-well microplates and incubated at 37° C. Each cell was incubated with a sample to induce a response and then diluted to generate a 5× sample in assay buffer. 5 μL of the 5× sample was then added to cells and incubated at 37° C. for 90 or 180 minutes. The final assay concentration was 1%. The assay signal was generated through addition of 12.5 μL or 15 μL (50% V/V) of a detection reagent cocktail followed by a one hour incubation time at room temperature. Microplates were read following signal generation with a PerkinElmer Envision™ (PerkinElmer Inc., Waltham, Mass.) for chemiluminescent signal detection. Reactivity was analyzed using the CBIS data analysis suite (ChemInnovation, CA) where RLU refers to the raw measured values. The percent activity was then calculated using the formula: % Activity=100%*(mean RLU of test sample−mean RLU of control)/(mean max control ligand−mean RLU of vehicle control).

In each of the antagonist assays, PathHunter® cell lines were removed from a freezer stock and seeded at a volume of 20 μL into white walled, 384-well microplates and incubated at 37° C. Each cell was pre-incubated with an antagonist followed by an agonist challenge at the EC80 concentration. Cells were then diluted to generate a 5× sample in assay buffer. 5 μL of the 5× sample was then added to cells and incubated at 37° C. for 30 minutes. Finally, 5 μL of 6×EC80 agonist in assay buffer were added to the cells and incubated at 37° C. for 90 minutes or 180 minutes. The assay signal was generated through addition of 12.5 μL or 15 μL (50% V/V) of a detection reagent cocktail followed by a one hour incubation time at room temperature. Microplates were read following signal generation with a PerkinElmer Envision™ (PerkinElmer Inc., Waltham, Mass.) for chemiluminescent signal detection. Reactivity was analyzed using the CBIS data analysis suite (ChemInnovation, CA) where RLU refers to the raw measured values. The percent inhibition was calculated using the formula: % Inhibition=100%*(1−mean RLU of test sample−mean RLU of control)/(mean RLU of EC80 control−mean RLU of vehicle control).

The results of the GCPR reactivity screen are depicted in Table 1. For agonist activity, the percent activity is depicted while for antagonist activity, the percent inhibition is depicted. As used herein, strong activity against a receptor is considered a percent activity of about 42% or higher. Higher results for antagonism against CHRM2, EDGE, and MRGPRX2 are considered better. It is further considered better when a cannabinoid inhibits more than one of the receptors.

TABLE 1

| | (+)-CBD | CBD | CBDV | CBD-C2 | CBD-C1 | CBG | CBG-C4 | CBGV | CBC | CBL |
|---|---|---|---|---|---|---|---|---|---|---|
| CHRM2 | 7% | 12% | 3% | 1% | −1% | 0% | 0% | 0% | 12% | 13% |
| (Agonist) | | | | | | | | | | |
| CHRM2 | 67% | 81% | 34% | 21% | 2% | 20% | 13% | 28% | 89% | 84% |
| (Antagonist) | | | | | | | | | | |
| EDG3 | 21% | 2% | 1% | 1% | 1% | 8% | 3% | 2% | −1% | −1% |
| (Agonist) | | | | | | | | | | |
| EDG3 | 55% | 35% | 24% | 20% | 13% | 42% | 28% | 29% | 48% | 9% |
| (Antagonist) | | | | | | | | | | |
| MRGPRX2 | 9% | 11% | 3% | 1% | 1% | 2% | 3% | 1% | 9% | 11% |
| (Agonist) | | | | | | | | | | |
| MRGPRX2 | 92% | 85% | 71% | 31% | 12% | 96% | 94% | 95% | 89% | 82% |
| (Antagonist) | | | | | | | | | | |

| | CBN | THCV | CBDA | CBDVA | CBGA | CBGA-C4 | CBGVA | CBCA | CBLA |
|---|---|---|---|---|---|---|---|---|---|
| CHRM2 | 0% | 18% | 1% | 2% | −3% | −4% | −4% | −1% | −4% |
| (Agonist) | | | | | | | | | |
| CHRM2 | 7% | 42% | −10% | 2% | 12% | 19% | 23% | 7% | −19% |
| (Antagonist) | | | | | | | | | |
| EDG3 | 5% | 1% | −1% | −3% | 2% | 0% | −1% | 1% | 3% |
| (Agonist) | | | | | | | | | |
| EDG3 | 12% | 20% | 5% | 13% | 12% | 16% | 9% | 32% | 19% |
| (Antagonist) | | | | | | | | | |
| MRGPRX2 | 22% | 18% | 4% | 5% | 0% | −1% | −1% | 4% | 2% |
| (Agonist) | | | | | | | | | |
| MRGPRX2 | 73% | 55% | 8% | −8% | 17% | 20% | 7% | 49% | 36% |
| (Antagonist) | | | | | | | | | |

As depicted in Table 1, none of the 19 evaluated cannabinoid compounds acted as an agonist for any of the three evaluated GPCR receptors. (+)-CBD and CBC each demonstrated strong to moderate antagonist activity against each of CHRM2, EDG3, and MRGPRX2 meaning they demonstrate strong potential to alleviate, or ameliorate, itching sensations. CBD and CBL each demonstrated antagonist activity for 2 of the receptors (CHRM2 and MRGPRX2) while CBDV, CBG, CBG-C4, CBGV, CBN, THCV, and CBCA each demonstrated antagonist activity against only MRGPRX2.

Based on the results of the GPCR reactivity screen, and IC₅₀ antagonist activity screen was run against each of CHRM2, EDG3, and MRGPRX2. The IC₅₀ antagonist activity screen was run for each of CBG, CBC, CBL, THCV, CBN, CBDV, CBD, CBD-C2, (+)-CBD, CBGV, CBG-C4, CBCA, CBG-C6, CBG-C7, CBG-C9, CBD-C6, CBD-C7, CBD-C9, CBGA-C6, CBGA-C7, and CBGA-C9. IC₅₀ curves were measured using the same GPCR assay, PathHunter® β-Arrestin from Eurofins DiscoverX Products (Fremont, Calif.), as the GPCR reactivity screen. The maximum inhibition (antagonism) for each of the cannabinoids is reported in Table 2. As used herein, strong activity against a receptor is considered a maximum activity of about 42% or higher. Higher results for antagonism against CHRM2, EDGE, and MRGPRX2 are considered better. It is further considered better when a cannabinoid inhibits more than one of the receptors.

TABLE 2

| Cannabinoid | CHRM2 | EDG3 | MRGPRX2 |
|---|---|---|---|
| CBG | 13 | 36 | 92 |
| CBC | 85 | 43 | 91 |
| CBL | 47 | — | 81 |
| THCV | 0 | — | 21 |
| CBN | — | — | 78 |
| CBDV | 27 | — | 59 |
| CBD | 75 | 30 | 96 |
| CBD-C2 | — | — | 43 |
| (+)-CBD | 62 | 24 | 94 |
| CBGV | — | — | 76 |
| CBG-C4 | — | — | 85 |
| CBCA | — | 25 | 35 |
| CBLA | — | — | 44 |
| CBG-C6 | 0 | 15 | 66 |
| CBG-C7 | 10 | 32 | 99 |
| CBG-C9 | 32 | 55 | 100 |
| CBD-C6 | 64 | 11 | 98 |
| CBD-C7 | 90 | 20 | 98 |
| CBD-C9 | 90 | 0 | 96 |
| CBGA-C6 | 6 | 33 | 13 |
| CBGA-C7 | 4 | 30 | 37 |
| CBGA-C9 | 11 | 58 | 56 |

The results depicted in Table 2 confirm the GPCR screening assay and confirm that cannabinoids including CBG, CBC, CBL, CBN, CBDV, and CBD exhibit an antagonistic effect on one or more of the receptors CHRM2, EDG3, and MRGPRX2. Although THCV demonstrated antagonism to MRGPRX2 in the GPRC screening assay, inhibition was not observed in the IC₅₀ data. Additionally, the IC₅₀ data shows that variants of such cannabinoids can also exhibit an antagonistic effect including (+)-CBD, CBD-C4, CBG-C6, CBG-C7, CBG-C9, CBD-C6, CBD-C7, CBD-C9, and CBGA-C9. In particular, the increasing alkyl side chain length is generally observed to increase the degree of antagonism to the GPCR receptor targets.

The dimensions and values disclosed herein are not to be understood as being strictly limited to the exact numerical values recited. Instead, unless otherwise specified, each such dimension is intended to mean both the recited value and a functionally equivalent range surrounding that value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in the document shall govern.

The foregoing description of embodiments and examples has been presented for purposes of description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent articles by those of ordinary skill in the art. Rather it is hereby intended the scope be defined by the claims appended hereto.

It should be understood that certain aspects, features, structures, or characteristics of the various embodiments can be interchanged in whole or in part. Reference to certain embodiments mean that a particular aspect, feature, structure, or characteristic described in connection with certain embodiments can be included in at least one embodiment and may be interchanged with certain other embodiments. The appearances of the phrase “in certain embodiments” in various places in specification are not necessarily all referring to the same embodiment, nor are certain embodiments necessarily mutually exclusive of other certain embodiments. It should also be understood that the steps of the methods set forth herein are not necessarily required to be performed in the orders described, and the order of the steps of such methods should be understood to be merely exemplary. Likewise, additional steps can be included in such methods, and certain steps may be omitted or combined, in methods consistent with certain embodiments.

What is claimed is:

1. A composition comprising one or more cannabinoid compounds, the composition or article alleviating the sensation of itching.

2. The composition of claim 1, wherein the one or more cannabinoid compounds comprise cannabidol (“CBD”), cannabichromene (“CBC”), cannabigerol (“CBG”), cannabinol (“CBN”), cannabicyclol (“CBL”), cannabidivarin (“CBDV”), cannabigerivarin (“CBGV”), and cannabicyclolic acid (“CBLA”) and variants thereof.

3. The composition of claim 2, wherein the variants thereof comprise (+)-cannabidiol (“(+)-CBD”), cannabigerol butyl (“CBG-C4”), cannabigerol hexyl (“CBG-C6”), cannabigerol heptyl (“CBG-C7”), cannabigerol nonyl (“CBG-C9”), cannabidol hexyl (“CBD-C6”), cannabidol heptyl (“CBD-C7”), cannabidol nonyl (“CBD-C9”), and cannabigerolic acid nonyl (“CBGA-C9”).

4. The composition of claim 1, wherein the one or more cannabinoid compounds are antagonists to one or more of cholinergic muscarinic receptor 2 (“CHRM2”), sphingosine 1-phosphate receptor 3 (“EDG3”), and MAS-Related GPR Family Member X2 (“MRGPRX2”).

5. The composition of claim 1, wherein the one or more cannabinoid compounds comprise CBC, CBD, and CBG.

6. The composition of claim 1 comprises about 100 mg or less of the one or more cannabinoid compounds.

7. The composition of claim 1 is topically applied to a desired location of a consumer's skin to alleviate the sensation of itching.

8. The composition of claim 1 is a powder or liquid.

9. The composition of claim 1 is a bath salt.

10. The composition of claim 1 is a personal care product.

11. The composition of claim 1 is a food or beverage.

12. The composition of claim 1 is a pill or capsule.

13. The composition of claim 12 is a delayed-release pill or capsule.

14. The composition of claim 1, wherein the one or more cannabinoid compounds are non-horticulturally derived cannabinoid compounds.

15. An article comprising the composition of claim 1.

16. A method of alleviating itching, the method comprising administering a therapeutically effective amount of one or more cannabinoid compounds.

17. The method of claim 16, wherein the one or more cannabinoid compounds comprise cannabidol (“CBD”), cannabichromene (“CBC”), cannabigerol (“CBG”), cannabinol (“CBN”), cannabicyclol (“CBL”), cannabidivarin (“CBDV”), cannabigerivarin (“CBGV”), and cannabicyclolic acid (“CBLA”), (+)-cannabidiol (“(+)-CBD”), cannabigerol butyl (“CBG-C4”), cannabigerol hexyl (“CBG-C6”), cannabigerol heptyl (“CBG-C7”), cannabigerol nonyl (“CBG-C9”), cannabidol hexyl (“CBD-C6”), cannabidol heptyl (“CBD-C7”), cannabidol nonyl (“CBD-C9”), and cannabigerolic acid nonyl (“CBGA-C9”).

18. The method of claim 16 treats acute itching.

19. The method of claim 16 treats chronic itching.

20. The method of claim 16 wherein the sensation of itching is substantially or fully alleviated.
